(19) United States
(12) Patent Application Publication
(10) Pub. No.: US 2024/0358290 A1
(43) Pub. Date: Oct. 31, 2024

(54) Portable kit and device for detecting or identifying diseases or disorders in a human subject by collecting biological sample from the human subject

(71) Applicant: AINOS, INC., San Diego, CA (US)

(72) Inventors: Chia-Nan LIAO, Zhubei City (TW); Chun-Hsien TSAI, Zhubei City (TW); Ting-Chuan LEE, Zhubei City (TW); Chun-Jung TSAI, Zhubei City (TW)

(21) Appl. No.: 18/771,339

(22) Filed: Jul. 12, 2024

Related U.S. Application Data
(63) Continuation-in-part of application No. 17/227,126, filed on Apr. 9, 2021.

(30) Foreign Application Priority Data
Apr. 9, 2020 (TW) 109112037

(51) Int. Cl. A61B 5/145; A61B 10/00

(52) U.S. Cl. CPC A61B 5/14546; A61B 5/14507; A61B 10/0045; A61B 2560/0462

(57) ABSTRACT

A device for detecting or identifying diseases or disorders in a human subject by collecting biological sample from the human subject comprises a pump unit, a first unit, a second unit, a gas permeable filter, and a collector. The first unit comprises a first chamber and a gas sensor unit. The second unit comprises a second chamber, an outlet portion and an inlet portion. The gas permeable filter is disposed in an upstream region of the second chamber. The collector is disposed in a downstream region of the second chamber. The pump unit is configured, when activated, to create a negative pressure in a gas flow pathway provided between the first chamber and second chamber through the outlet portion, thereby allowing an ambient gas stream forcedly flows from an exterior through the inlet portion into the second chamber and the first chamber.

### CROSS REFERENCING

This application is a continuation-in-part of U.S. Nonprovisional patent application Ser. No. 17/227,126, filed Apr. 9, 2021, which claims the benefit of Taiwanese Application No. 109112037, filed on Apr. 9, 2020, the contents of which are relied upon and incorporated herein by reference in their entirety.

### FIELD OF THE INVENTION

The present invention is related to the field of bio/chemical sensing, assays and applications.

### BACKGROUND OF THE INVENTION

The majority of clinical decisions rely on laboratory and health test data. However, the testing process may take days to weeks to complete. Accessing accurate testing information earlier during patient triage allows for earlier interventions and better disease management, ultimately improving outcomes and reducing care costs.

There is a need to conduct analyte analysis in the field or without requiring expensive and cumbersome support equipment typically found in hospitals, laboratories, or test facilities. In many cases, it is desirable to use a largely self-contained, portable, and easy-to-use device. It is also necessary or desirable in some instances to have the capability to detect the analyte in the fluid stream in real-time or near real-time. Moreover, it is crucial to perform such sensing accurately and reliably.

### SUMMARY OF THE INVENTION

The present disclosure provides for a portable device for detecting or identifying diseases or disorders in a human subject by collecting biological sample from the human subject. The device comprises a pump unit, a first unit, a second unit, a gas permeable filter and a collector. The first unit comprises a first chamber fluidly connecting to the pump unit and a gas sensor unit disposed in the first chamber. The second unit comprises a second chamber, an outlet portion fluidly connecting between the second chamber and the first chamber, and an inlet portion fluidly connecting between the second chamber and an exterior of the second chamber. The pump unit is configured, when activated, to create a negative pressure in a gas flow pathway provided between the first chamber and second chamber through the outlet portion, thereby allowing an ambient gas stream forcedly flows from the exterior through the inlet portion into the second chamber and the first chamber. The gas permeable filter is disposed in an upstream region of the second chamber. The collector is disposed in a downstream region of the second chamber. The collector is configured to temporarily secure a biological sample of a human subject. A purified gas stream is produced by suction of the ambient gas stream passing through the gas permeable filter and carries volatile compounds of the biological sample from the second chamber to the first chamber.

### BRIEF DESCRIPTION OF THE DRAWINGS

The accompanying drawings that are incorporated in and constitute a part of this specification, illustrate several embodiments of the disclosure and, together with their descriptions, serve to explain the principles of the disclosure.

FIG. 1 shows a perspective view of the portable kit according to the invention;

FIG. 2 shows a side plan view of the portable test device of FIG. 1;

FIG. 3 shows an exploded view of the portable test device of FIG. 1;

FIG. 4 shows an exploded view of the cartridge of FIG. 1;

FIG. 5 shows a detailed view of a portion of the portable test device;

FIG. 6 shows a detailed view of a portion of the portable test device; and

FIGS. 7A-7D show a process of analyzing the biological sample according to the invention.

### DETAILED DESCRIPTION OF THE PREFERRED EMBODIMENTS

Reference will now be made in detail to the presently preferred embodiments and methods of the invention as described herein below and as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

The present invention relates to portable devices and kits for detecting or identifying diseases or disorders in a human subject by capturing a biological sample from the human subject. In one example, the devices and kits preferably senses of analytes of one or more biological sample from a human subject. The biological sample may be a bodily fluid or secretion, such as exhale, breath, vaginal discharge, blood, serum, urine, saliva, spinal fluid, sweat, tears, vaginal fluid, mucous, or semen.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

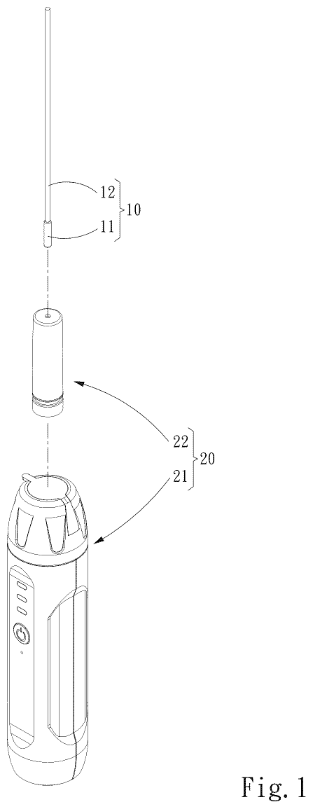

FIG. 1 is an exemplary embodiment of a portable kit according to certain aspects of the invention for analyzing at least one analyte in a bodily fluid or secretion. The kit of FIG. 1 comprises a collection tool 10 and a test module 20.

The collection tool 10 comprise an absorbent portion 11 and a stem 12. The absorbent portion 11 attaches to the stem 12. The absorbent portion 11 (sometimes referred to as collector) is configured to collect or temporarily secure a biological sample of a human subject. In the present example, the collection tool 10 may be a swab.

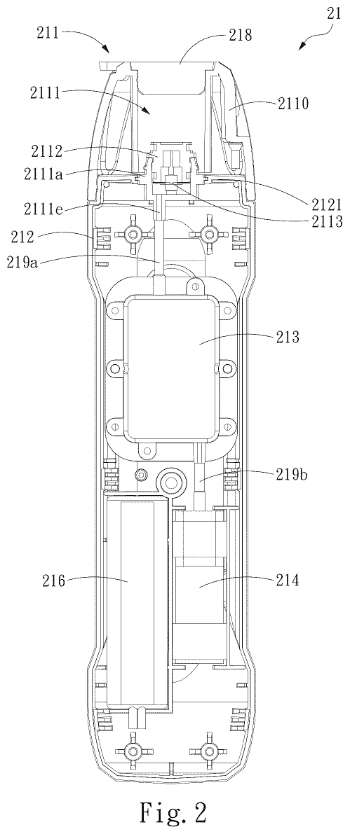

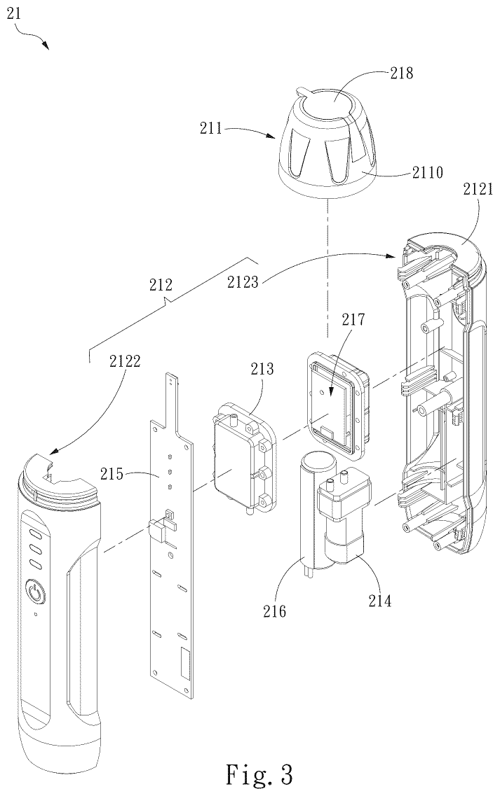

Referring to FIG. 2 and FIG. 3, the test module 20 comprises a portable test device 21 and a cartridge 22. The portable test device 21 includes a head portion 211, a casing 212, a chamber 213 (sometimes referred to as a second unit and an interior space of the chamber 213 referred to as a second chamber), a pump 214 (sometimes referred to as a pump unit), a controller 215 and a battery component 216. The head portion 211 is mounted on a top end 2121 of the casing 212 and the casing 212 have a first half portion 2122 and a second half portion 2123. The chamber 213, the pump 214, the controller 215 and the battery component 216 are disposed inside the casing 212. The portable test device 21 further includes a sensor unit 217 disposed in the chamber 213. The sensor unit 217 may be a gas sensor circuit including one or more gas sensors. The gas sensor may be a solid-state semiconductor sensor, which is adaptable to react with the biological sample from the human subject. The sensor unit 217 is connected to the controller 215. The controller 215 includes a processor to determine if the reaction between the gas sensor and the biological sample matches a result associated to one or more diseases or disorders. The pump 214 operably connects to the chamber 213, for creating negative pressure in the chamber 213.

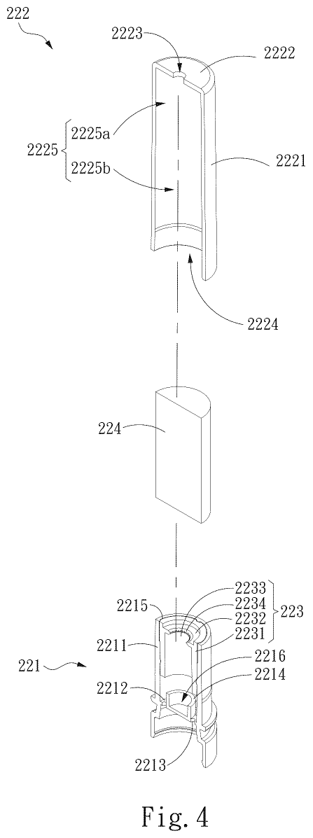

Referring to FIG. 4, the cartridge 22 (sometimes referred to as a first unit and an interior space in the cartridge 22 referred to as a first chamber) is used for retention of the biological sample. The cartridge 22 may comprise a base 221 (sometimes referred to as an outlet portion), a cap 222 (sometimes referred to as an inlet portion), a cutting component 223 and a gas permeable filter 224. The base 221 includes a housing 2211, a bottom baffle 2212, one or more venting holes 2213, a tubular wall 2214 and a top opening 2215. The housing 2211 is substantially cylindrical and oriented longitudinally. The bottom baffle 2212 is formed as a disc shape and disposed in the housing 2211. The bottom baffle 2212 has a base portion and an outer periphery. The tubular wall 2214 extended upright from a perimeter of the base portion, thereby defining a cavity 2216 therein. The venting holes 2213 are provided on the outer periphery of the bottom baffle 2212. The cutting component 223 is provided on the top opening 2215 and above the tubular wall 2214, such that the cavity 2216 is exposed within the base 221.

The cutting component 223 comprises a sleeve 2231, a collar 2232, an axial bore 2233 and a blade structure 2234. The sleeve 2231 is configured to be received within the housing 2211. The blade structure 2234 is formed on a circumferential edge of the axial bore 2233. The blade structure 2234 may comprise a plurality of saws formed along the circumferential edge of the axial bore 2233. The blade structure 2234 is used for breaking the collection tool 10 to leave the absorbent portion 11 in the cavity 2216, as will be described later.

Additionally, the cavity 2216 is intended to be aligned with the axial bore 2233 to facilitate receiving the collection tool 10 after cutting. By arranging the venting holes 2213 on the outer periphery of the bottom baffle 2212, spillage of the biological sample through the venting holes 2213 can be avoided.

The cap 222 may comprise a body 2221, a top portion 2222, an aperture 2223 and a bottom opening 2224. The body 2221 together with the top portion 2222 may be collectively referred to as an enclosure. The body 2221 is substantially cylindrical and defines an interior space 2225. The aperture 2223 is formed on the top portion 2222, which allows a gas or fluid to pass through them. The interior space 2225 has a first region 2225a and a second region 2225b, the first region 2225a is proximity to the top portion 2222 and the second region 2225b is proximity to the bottom opening 2224. The first region 2225a may be defined as an upstream region of the second chamber and the second region 2225b may be defined as a downstream region of the second chamber.

The gas permeable filter 224 may be formed in a barrel-shaped and is fitted in the first region 2225a. Furthermore, the gas permeable filter 224 may be inserted into the first region 2225a and cover over the aperture 2223. In one example, the gas permeable filter 224 is a carbon filter cartridge.

The cap 222 is configured to releasably cover the base 221 and can be snug fitted, or otherwise attached to the base 221, to form a substantially liquid-tight and gas-tight seal between the cap 222 and the base 221. When the cap 222 is mounted on the base 221, the tubular wall 2214 and the cutting component 223 are received in the second region 2225b of the cap 222.

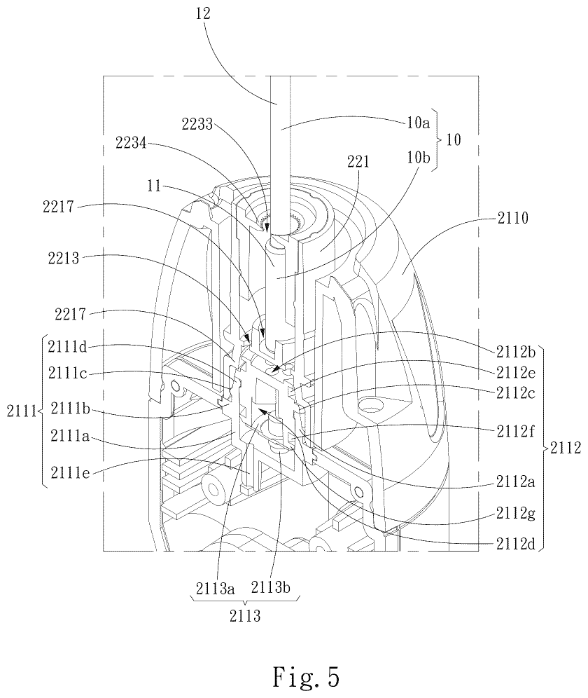

The cartridge 22 is used to retain the absorbent portion 11 of the collection tool 10. In the present example, the collection tool 10 is a swab and the biological sample is attached onto or absorbed into the absorbent portion 11. Before testing, the cap 222 is separated from the base 221 and the swab 10 is disposed in the base 221. As illustrated in FIG. 5, the swab 10 is inserted into the axial bore 2233, leaving a sacrifice section 10a of the swab 10 is above the axial bore 2233 and a remaining section 10b (sometimes referred to as collector) of the swab 10 is below the axial bore 2233. The remaining section 10b includes at least a portion of the absorbent portion 11. When moving the stem 12 of the swab 10, the swab 10 is cut by the blade structure 2234 and then the remaining section 10b is broken away from the sacrifice section 10a. The axial bore 2233 is coaxial aligned with the cavity 2216, so the remaining section 10b is fall in the cavity 2216 after broken. Subsequently, the cap 222 is mounted on the base 221.

The liquid-tight and gas-tight connection between the cap 222 and the base 221 allows flowing of a gas from the aperture 2223 through the gas permeable filter 224 and down to the venting holes 2213. By flowing of a gas stream or an ambient gas stream in a chamber defined in the cap 222 and the base 221, the biological sample attached onto or absorbed in the absorbent portion 11 can be delivered with the gas stream.

In the present example, the cartridge 22 is configured to be assembled with the portable test device 21. In some instance, the cap 222 together with the gas permeable filter 224 may be solely assembled with the portable test device 21, without the base 221 and the cutting component 223, which will be described later.

The head portion 211 of the portable test device 21 is sized and shaped to receive the cartridge 22. As shown in FIG. 2, the head portion 211 has a tube portion 2110, a socket 2111, a valve 2112 and a stopper 2113. The tube portion 2110 comprises a hollow to mate with the base 221 of the cartridge 22. The portable test device 21 may further include a removable cover 218 connecting to the tube portion 2110. The socket 2111 is provided within the hollow. The socket 2111 includes a holder 2111a, a peripheral extension 2111b, an outer flange 2111c, a top rim 2111d and a connecting conduit 2111e. The holder 2111a is formed as a cylindrical depression to operatively couple with the valve 2112. The peripheral extension 2111b and the outer flange 2111c are protruded outward radially from the holder 2111a, which may be substantially annular in shape. The peripheral extension 2111b adaptively attaches to the first half portion 2122 and the second half portion 2123 of the casing 212. The outer flange 2111c adaptively attaches to a lower receptacle 2217 of the base 221. The connecting conduit 2111e opens to the cylindrical depression of the holder 2111a and connects fluidly to the chamber 213 through a first connecting tube 219a. The chamber 213 connects fluidly to the pump 214 through a second connecting tube 219b as shown in FIG. 2.

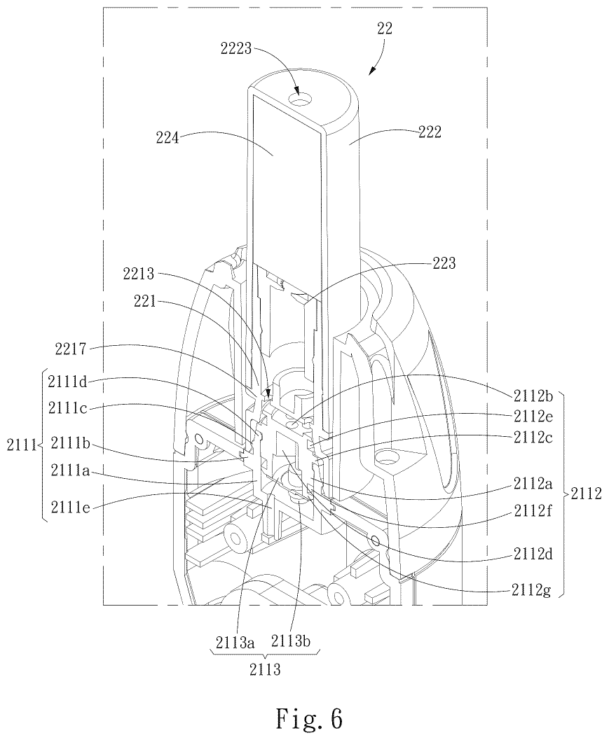

Referring to FIG. 6, when the lower receptacle 2217 of the base 221 is assembled with the outer flange 2111c of the socket 2111, an annular chamber is provided between a lower periphery of the base 221 and an upper periphery of the socket 2111 and an annular gap is provided between a step in the lower receptacle 2217 and a top rim 2111d of the socket 2111. The valve 2112 is disposed between the lower receptacle 2217 of the base 221 and the holder 2111a. The valve 2112 has a valve body 2112a, one or more inlets 2112b, an annular rib 2112c, a lower cavity 2112d, an upper recess 2112e and a lower recess 2112f. The valve body 2112a is formed in a tubular shape which is configured to mate with the cylindrical depression of the holder 2111a.

The inlets 2112b are axially through a central portion of the valve body 2112a and in fluid communication with the lower cavity 2112d. The annular rib 2112c extends outwardly around the outer periphery of the valve body 2112a, which is disposed within the annular gap between the step in the lower receptacle 2217 and the top rim 2111d of the socket 2111. The upper recess 2112e and the lower recess 2112f are provided with O-rings to ensure liquid-tight and gas-tight seal between the valve body 2112a and the base 221 and the socket 2111.

The stopper 2113 is disposed in the socket 2111 and between a bottom wall of the socket 2111 and the lower cavity 2112d. The stopper 2113 includes a circular flap 2113a and a stud portion 2113b. The stud portion 2113b attaches to a bottom wall of the socket 2111 and the circular flap 2113a is disposed on the stud portion 2113b. The circular flap 2113a has a diameter larger than an opening of the lower receptacle 2217 and the stud portion 2113b has a diameter smaller than the opening of the lower receptacle 2217, thereby the circular flap 2113a is suspended in the socket 2111.

The circular flap 2113a is made of an elastically deformable material. When the pump 214 is turned off, and no negative pressure exists in the chamber 213 (or in the cylindrical depression of the holder 2111a), the circular flap 2113a seals and closes the opening of the lower receptacle 2217 (in a normal state). That is, fluid communication between the cylindrical depression of the holder 2111a and the lower cavity 2116 of the valve 2112 is closed.

Upon the negative pressure generated in the chamber 213 by turning on the pump 214, the suction force causes the circular flap 2113a to bend down, creating a circular gap between the lower receptacle 2217 and the deformed circular flap 2113a. This opens the lower receptacle 2217, thereby permitting fluid communication between the cylindrical depression of the holder 2111a and the lower cavity 2116 of the valve 2112.

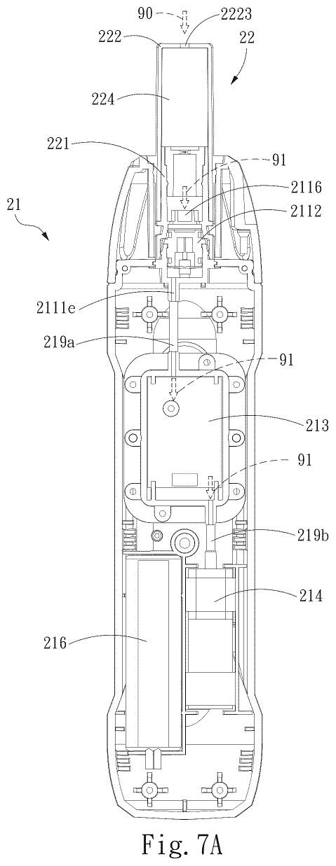

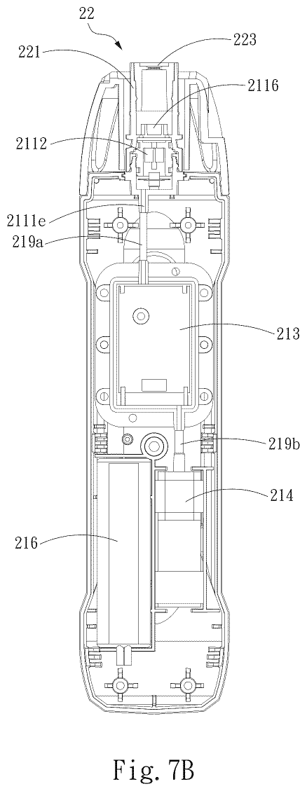

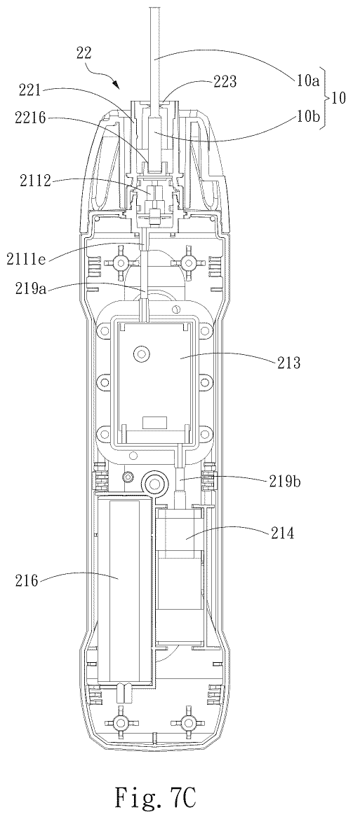

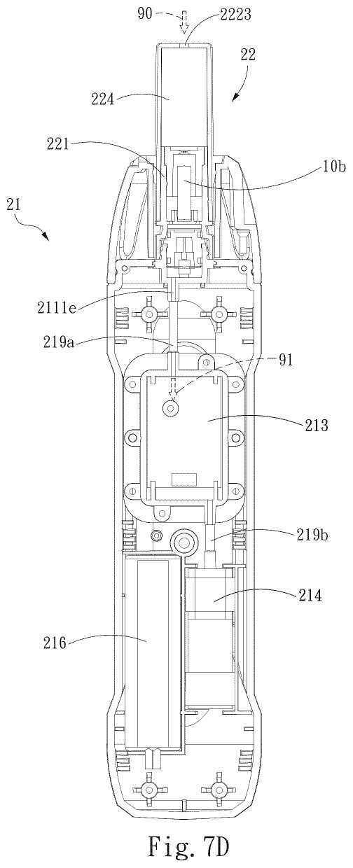

Below is the detailed process of a method for a diagnostic test according to an embodiment. In operation, a pre-cleaning step is performed first. Referring to FIG. 7A, the cartridge 22 without the biological sample stored inside is engaged with the portable test device 21. By inserting the base 221 into the socket 2111, the cavity 2216, the venting holes 2213, the lower receptacle 2217, and the valve 2112 are in fluid communication along the longitudinal direction. The valve 2112 is therefore in fluid communication with the lower cavity 2116 of the valve 2112 and the connecting conduit 2111e. After installed, a gas flow pathway is established from the aperture 2223 of the cap 222, through the gas permeable filter 224, the venting holes 2213, the inlet 2114 of the valve 2112, the connecting conduit 2111e, to the chamber 213. Actuating the pump 214 causes outside air 90 to be drawn into the chamber 213 through the gas flow pathway. The air is purified after passing through the gas permeable filter 224, then purified air 91 (or gas stream) flows over the gas flow pathway by a suction force produced by the pump 214. After the pump 214 turned off, the purified air stays in the chamber 213 to ensure the environment for testing maintains no or a very low concentration of airborne particulates or contaminations.

Alternatively, during the pre-cleaning step, the base 221 and the cutting component 223 shown in FIG. 7A are not required. In other words, the pre-cleaning step may involve only the cap 222 and the gas permeable filter 224. The gas permeable filter 224 is placed into the cap 222, which is then attached to the portable test device 21.

After the pre-cleaning step, the biological sample is taken from a human subject. The biological sample may be obtained by a technique selected from scrapes, swabs, and biopsy. In embodiments, the biological sample is obtained by use of the collection tool 10 such as brushes, (cotton) swabs, spatula, rinse/wash fluids, punch biopsy devices, puncture of cavities with needles or surgical instrumentation. Once the collection tool 10 receives the biological sample from the human subject, remove the cap 222 from the base 221 (as shown in FIG. 7B), and then insert the collection tool 10 into the base 221 of the cartridge 22, as shown in FIG. 7C.

Then breaking the collection tool 10 as previously described to leave the remaining section 10b in the cavity 2216 of the base 221. After the remaining section 10b containing the absorbent portion 11 is retained within the base 221, the cap 222, fitted with the gas permeable filter 224, is mounted on the base 221 to assemble the cartridge 22, as shown in FIG. 7D.

Next, actuating the pump 214 again. The outside air is drawn into the chamber 213 through the gas flow pathway. Then volatile compounds in the biological sample, for instance the volatile organic compounds (VOCs), flows into the chamber 213 with the purified air stream.

The compounds in the biological sample can react with the gas sensor and the reaction can be analyzed by the processor to determine if the reaction between the gas sensor and the biological sample matches a result associated to one or more diseases or disorders.

One advantage of the present disclosure is that the analysis of biological samples can be performed in clean environments due to the pre-cleaning step conducted prior to analysis. Additionally, the influence of ambient air noise is minimized by arranging the filter in the gas flow pathway and placing the biological sample inside the cartridge. The releasable and replaceable cartridge also facilitate point-of-care testing.

What is claimed is:

1. A portable device for detecting or identifying diseases or disorders in a human subject by collecting biological sample from the human subject, comprising:
a pump unit;
a first unit comprising a first chamber fluidly connecting to the pump unit and a gas sensor unit disposed in the first chamber;
a second unit comprising a second chamber, an outlet portion fluidly connecting between the second chamber and the first chamber, and an inlet portion fluidly connecting between the second chamber and an exterior of the second chamber, wherein the pump unit is configured, when activated, to create a negative pressure in a gas flow pathway provided between the first chamber and second chamber through the outlet portion, thereby allowing an ambient gas stream forcedly flows from the exterior through the inlet portion into the second chamber and the first chamber;
a gas permeable filter disposed in an upstream region of the second chamber; and
a collector disposed in a downstream region of the second chamber, wherein the collector is configured to temporarily secure a biological sample of a human subject;
wherein a purified gas stream is produced by suction of the ambient gas stream passing through the gas permeable filter and carries volatile compounds of the biological sample from the second chamber to the first chamber.

2. The device according to claim 1, wherein the gas permeable filter is a carbon filter cartridge.

3. The device according to claim 1, further comprising:
a casing, having a head portion and a socket formed on the head portion;
a valve, disposed in the socket of the casing; and
a controller;
wherein the first unit, the pump unit and the controller are disposed inside the casing; and
wherein the second unit is releasably engaged to the socket of the casing and fluidly connected to the second unit through the valve.

4. The device according to claim 3, wherein a removable cover is disposed on the socket when the second unit is disengaged with the socket of the casing.

5. The device according to claim 1, wherein the second unit further comprises:
a base, comprising a bottom baffle, one or more venting holes penetrating the bottom baffle, a tubular wall extended upright from the bottom baffle and defining a cavity therein and a top opening above the cavity, wherein the venting holes is fluidly connected with the first chamber; and
a cap mounted on the base to form the second chamber therein, comprising an enclosure defining an interior space, an aperture formed on a top portion of the enclosure and a bottom opening;
wherein the upstream region of the second chamber is adjacent to the aperture and the downstream region of the second chamber is adjacent to the upstream region, the gas permeable filter is disposed in the upstream region and closes off the aperture, the tubular wall is positioned within the downstream region;
wherein the cavity of the base is covered by the gas permeable filter from above and enclosed circumferentially by the enclosure.

6. The device according to claim 5, wherein a cutting component is provided on the top opening, the cutting component comprises an axial bore and a blade structure formed on a circumferential edge of the axial bore.

7. The device according to claim 6, wherein the aperture is coaxially aligned with the axial bore.

8. The device according to claim 7, wherein the collection tool is inserted into the axial bore such that the absorbent portion is passed through the axial bore and a section of the collection tool stays adjacent to the blade structure.

9. The device according to claim 8, wherein collection tool is configured to be cut by the blade structure into a sacrifice section and a remaining section containing at least a portion of the absorbent portion, when the collection tool is moved to the blade structure.

10. The device according to claim 9, wherein the remaining section is received in the cavity of the base after cutting.

11. A portable kit for detecting or identifying diseases or disorders in a human subject by collecting biological sample from the human subject, comprising:
a collection tool, comprising an absorbent portion and an elongate portion adjoining to the absorbent portion, wherein the absorbent portion is configured to temporarily secure a biological sample of a human subject;
a test module, comprising:
a pump unit;
a first unit comprising a first chamber fluidly connecting to the pump unit and a gas sensor unit disposed in the first chamber;
a second unit comprising a second chamber, an outlet portion fluidly connecting between the second chamber and the first chamber, an inlet portion fluidly connecting between the second chamber and an exterior of the second chamber, wherein the pump unit is configured, when activated, to create a negative pressure in a gas flow pathway provided between the first chamber and second chamber through the outlet portion, thereby allowing an ambient gas stream forcedly flows from the exterior through the inlet portion into the second chamber and the first chamber;
a gas permeable filter disposed in an upstream region of the second chamber;
wherein at least a portion of the absorbent portion of the collection tool is disposed in a downstream region of the second chamber; and
wherein a purified gas stream is produced by suction of the ambient gas stream passing through the gas permeable filter and carries volatile compounds of the biological sample from the second chamber to the first chamber.

12. The kit according to claim 11, wherein the gas permeable filter is a carbon filter cartridge.

13. The kit according to claim 11, further comprising:
a casing, having a head portion and a socket formed on the head portion;
a valve, disposed in the socket of the casing; and
a controller;
wherein the first unit, the pump unit and the controller are disposed inside the casing; and
wherein the second unit is releasably engaged to the socket of the casing and fluidly connected to the second unit through the valve.

14. The kit according to claim 11, wherein the second unit further comprises:
a base, comprising a bottom baffle, one or more venting holes penetrating the bottom baffle, a tubular wall extended upright from the bottom baffle and defining a cavity therein and a top opening above the cavity, wherein the venting holes is fluidly connected with the first chamber; and
a cap mounted on the base to form the second chamber therein, comprising an enclosure defining an interior space, an aperture formed on a top portion of the enclosure and a bottom opening;
wherein the upstream region of the second chamber is adjacent to the aperture and the downstream region of the second chamber is adjacent to the upstream region, the gas permeable filter is disposed in the upstream region and closes off the aperture, the tubular wall is positioned within the downstream region;
wherein the cavity of the base is covered by the gas permeable filter from above and enclosed circumferentially by the enclosure.

15. The kit according to claim 14, wherein a cutting component is provided on the top opening, the cutting component comprises an axial bore and a blade structure formed on a circumferential edge of the axial bore.

16. The kit according to claim 15, wherein the collection tool is inserted into the axial bore such that the absorbent portion is passed through the axial bore and a section of the collection tool stays adjacent to the blade structure.

17. The kit according to claim 16, wherein collection tool is configured to be cut by the blade structure into a sacrifice section and a remaining section containing at least a portion of the absorbent portion, when the collection tool is moved to the blade structure.

18. The kit according to claim 17, wherein the remaining section is received in the cavity of the base after cutting.
